(19) United States
(12) Patent Application Publication
(10) Pub. No.: US 2010/0192961 A1
(43) Pub. Date: Aug. 5, 2010

(54) TRACKABLE REFERENCE DEVICE FOR COMPUTER-ASSISTED SURGERY

(76) Inventors: Louis-Philippe Amiot, Hampstead (CA); Pierre Couture, Montreal (CA); Sébastien Jutras, Laval (CA); Daniel Odermatt, Montreal (CA); Emmanuel Daigneault, Montreal (CA); Ahmed Aoude, Pointe-Claire (CA)

Correspondence Address: OGILVY RENAULT LLP, 1, Place Ville Marie, SUITE 2500, MONTREAL, QC, H3B 1R1, CA

(21) Appl. No.: 12/670,151

(22) PCT Filed: Nov. 10, 2008
(86) PCT No.: PCT/CA08/01989

§ 371 Date: Mar. 15, 2010

Related U.S. Application Data
(60) Provisional application No. 60/986,374, filed on Nov. 8, 2007.
(60) Provisional application No. 61/100,827, filed on Sep. 29, 2008.

(51) Int. Cl. A61B 19/00; A61F 5/37

(52) U.S. Cl. 128/882; 606/130

(57) ABSTRACT

A surgical reference device for use with a computer-assisted surgery system comprises an engaging member adapted to engage directly on soft tissue of a body part such that substantially no relative movement is possible between the engaging member and a bone element of the body part. A trackable member is attached to the engaging member and comprises a detectable element for being located and tracked in three-dimensional space by the computer-assisted surgery system to measure a position and an orientation of the trackable member in time, whereby the bone element is tracked as a function of the position and orientation of the trackable member. A method for tracking a position and an orientation of a bone element using a computer-assisted surgery system is also provided.

### CROSS-REFERENCE TO RELATED APPLICATIONS

This patent application claims priority on U.S. Provisional Applications No. 60/986,374, filed on Nov. 8, 2007, and No. 61/100,827, filed on Sep. 29, 2008.

### FIELD OF THE APPLICATION

The present application relates to trackable references for use with a Computer-Assisted Surgery (CAS) system.

### BACKGROUND OF THE ART

CAS systems are used to track the locations of a plurality of markers present within a surgical field in real time, so as to provide navigational data relating bone elements to surgical tools. The markers are on a frame member which is typically fastened directly to each of the relevant bone elements to enable the CAS system to locate and track each of the bone elements, and hence define a frame of reference.

Reference members vary depending on the type and specific requirements of a particular CAS system in use. Reference members are nevertheless typically used to identify the position and the orientation in space of the bone element to which they are respectively attached.

Markers on each of the reference members are located and tracked by detectors such that their position and/or orientation is calculated by the CAS system.

Precise knowledge of the position and/or orientation of each marker therefore allows the determination of a position and/or orientation of the frame member, and consequently of the bone element to which it is affixed.

The above-described tracking scheme commonly requires that the frame member be directly anchored to the bone element using bone-anchoring methods. Such methods are not always possible or preferable, especially when the patient is afflicted by a degenerative bone disease, for example.

Moreover, because of the invasiveness of some currently-used reference members, some orthopedic surgeons have opted not to use computer-assisted surgery methods. Some reference markers typically leave anchoring holes in the bones, which may lead to patient discomfort and an increased risk of infection and fractures.

### SUMMARY OF THE APPLICATION

It is therefore an aim of the present application to provide a CAS reference device and tracking method that addresses issues associated with the prior art.

Therefore, in accordance with a first embodiment of the present application, there is provided a surgical reference device for use with a computer-assisted surgery system, the surgical reference device comprising: an engaging member adapted to engage directly on soft tissue of a body part such that substantially no relative movement is possible between the engaging member and a bone element of the body part; and a trackable member attached to the engaging member, the trackable member comprising a detectable element for being located and tracked in three-dimensional space by the computer-assisted surgery system to measure a position and an orientation of the trackable member in time; whereby the bone element is tracked as a function of the position and orientation of the trackable member.

Further in accordance with the first embodiment, the body part is a foot of a patient and the engaging member is a foot holder adapted to receive the foot of the patient, the trackable member being secured to the foot holder.

Still further in accordance with the first embodiment, the foot holder has a pair of malleoli contacting members adapted to releasably abut against opposite malleoli of the foot to immovably secure the foot holder to the foot of the patient.

Still further in accordance with the first embodiment the foot holder has means to abut against a diaphysis of a tibia of the patient to immovable secure the foot holder to the tibia of the patient.

Still further in accordance with the first embodiment, at least one of the malleoli contacting members is mounted to a shell of the foot holder by a threaded rod allowing translation displacement of the malleoli contacting member.

Still further in accordance with the first embodiment, the foot holder has at least one inflatable member adapted to immovably secure the foot between an outer shell of the foot holder and the inflatable member.

Still further in accordance with the first embodiment, the engaging member is a strap strapped to the body part, the strap being secured by Velcro™ strips.

Still further in accordance with the first embodiment, the body part is a thigh of the patient, and the strap is adapted to be immovably strapped to the thigh of the patient.

Still further in accordance with the first embodiment, the trackable member is connected to an engaging member by a lockable cylindrical joint.

Still further in accordance with the first embodiment, the surgical reference device is combined with a support table adapted to support the client; and at least one lockable joint between the surgical reference device and the support table to adjust at least one of a position and an orientation of the surgical reference device with respect to the table.

Still further in accordance with the first embodiment 10, the at least one joint comprises a lockable rotational joint and a lockable translational joint such that a position and an orientation of the surgical reference device is adjustable with respect to the support table.

In accordance with a second embodiment of the present application, there is provided a method for tracking a position and an orientation of a bone element using a computer-assisted surgery system, the method comprising: securing an engaging member directly on soft tissue of a body part such that substantially no relative movement is possible between the engaging member and a bone element of the body part, the engaging member having a trackable member thereon; performing a registration of an exposed portion the bone element with respect to the trackable member; and obtaining position and orientation data for at least the exposed portion of the bone element as a function of the tracking of a position and an orientation of the trackable member and the registration.

Further in accordance with the second embodiment, securing an engaging member directly on soft tissue of a body part comprises inserting a foot of the user into a foot support and securing the foot to the foot support.

Still further in accordance with the second embodiment, securing the foot to the foot support comprises abutting contacting members against the malleoli of the patient.

Still further in accordance with the second embodiment, securing the foot to the foot support further comprises securing the foot support against diaphysis of the tibia.

Still further in accordance with the second embodiment, securing the foot to the foot support comprises inflating an inflatable member such that the foot is secured between the inflatable member and the foot support.

Still further in accordance with the second embodiment, securing an engaging member directly on soft tissue of a body part comprises strapping the engaging member against a thigh of the patient.

Still further in accordance with the second embodiment, adjusting at least one of a position and an orientation of the foot holder with respect to a support table by adjusting at least one joint between the foot holder and the support table, prior to performing the registration, for the trackable member to be detectable by the computer-assisted surgery system.

Still further in accordance with the second embodiment, further comprising adjusting at least one of a position and an orientation of the trackable member with respect to the engaging member prior to performing the registration, for the trackable member to be detectable by the computer-assisted surgery system.

### BRIEF DESCRIPTION OF THE DRAWINGS

FIG. 1 is a perspective view of a surgical reference device to be fixed directly to a bone element in accordance with the prior art;

FIG. 2 is a schematic view of a surgical reference device in accordance with an embodiment of the present application;

FIG. 3 is a perspective view of the surgical reference device of FIG. 2 where an engaging member is a foot holder;

FIG. 4 is a perspective view of the surgical reference device of FIG. 2 where the engaging member is a compression bandage;

FIG. 5 is a flowchart of a method for tracking a position and an orientation of a bone element in accordance with an embodiment of the present application;

FIG. 6 is a schematic view of the surgical reference device of FIG. 2 where an engaging member is a foot holder with malleoli contacting members; and

FIG. 7 is a schematic view of the surgical reference device of FIG. 2 where an engaging member is a foot holder with an inflatable cushion.

### DESCRIPTION OF THE PREFERRED EMBODIMENTS

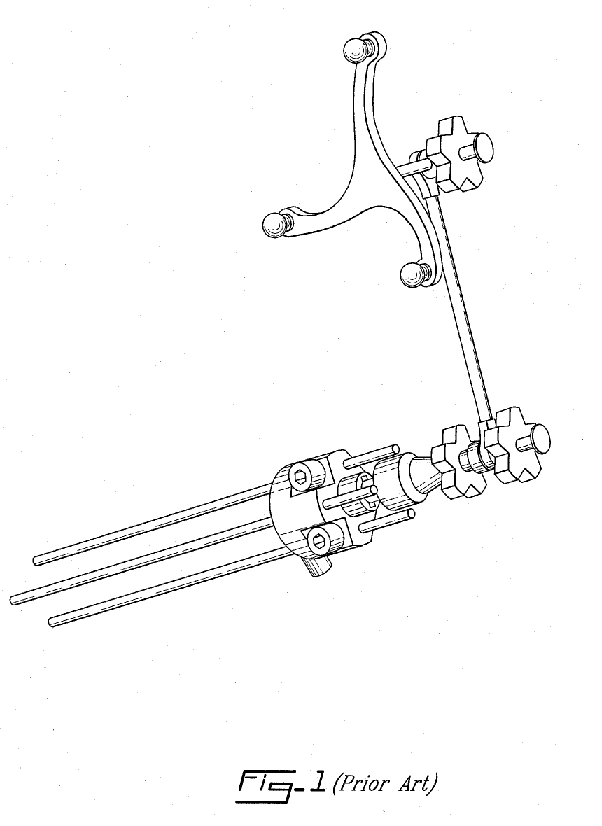

Unlike the prior-art surgical reference assembly illustrated by FIG. 1, which is made to be fixed directly to a bone of a patient, the present application proposes a surgical reference assembly and tracking method adapted to track a bone element within a body part of the patient without fixing anything to the bone.

The prior-art surgical bone reference assembly illustrated in FIG. 1 generally comprises a bone anchor member, an articulated support having a trackable member engaged at one end thereof, and is removably engageable and disengageable with the bone anchor member by an attachment member. The trackable member allows for the CAS system to track the position and orientation of the reference assembly and thus also of the bone having the bone anchor member anchored therein.

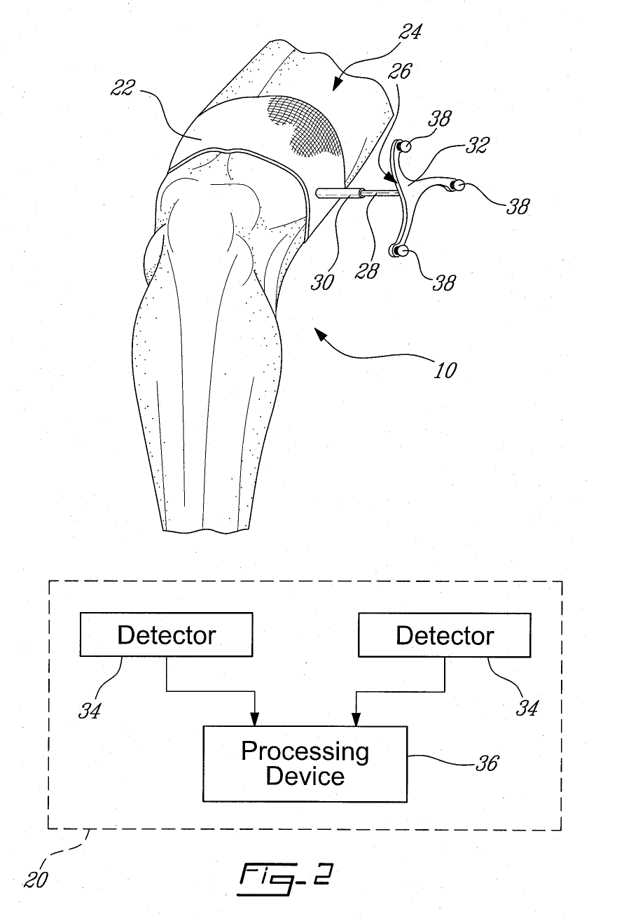

FIG. 2 schematically illustrates a surgical reference device 10 for use with a CAS system 20 which is located at a distance from a patient, for instance remotely from the surgical field.

The surgical reference device 10 has an engaging member 22 for engaging directly on soft tissue of a body part 24. The engaging member 22 can optionally be engaged partially around the body part 24 of a patient, on the soft tissue of the body part 24.

A trackable member 26 is attached to the engaging member 22.

An optional adjustable support member 28 can be used to attach member 26 to member 22.

Alternatively, both members 26 and 22 can define an integral member. In such a case, the engaging member 22 can be positioned on the soft tissue of the body part 24, with respect to a bone element within the body part, such that a position and an orientation of the trackable member 26 with respect to the bone element can be set as desired to be visible by sensors/detectors of a computer-assisted surgery system.

If the adjustable support member 28 is used, a first end of the adjustable support member 28 allows the variation of a position and/or an orientation of the trackable member 26 with respect to the adjustable support member 28 and the engaging member 22.

A second end of the adjustable support member is removably fastened, and thus detachable and refastenable, to the engaging member 22 via an attachment member 30.

The attachment member 30 therefore permits a removal of the trackable member 26 with the adjustable support member 28 from the engaging member 22, intraoperatively or extraoperatively. For instance, the adjustable support member 28 and the attachment member 30 define a lockable cylindrical joint, allowing the positional and orientational adjustment of the trackable member 26 with respect to the engaging member 22. The adjustable support member 28 may therefore allow the trackable member 26 to be affixed to the engaging member 22 in selected position and orientation relative to the bone element in the body part 24.

Still referring to FIG. 2, the engaging member 22 is made to engage on soft tissue of the body part 24 in such a way to substantially block any relative movement between the engaging member and a bone element of the body part 24.

The trackable member 26 of the surgical reference device 10 has a detectable element 32 for being located and tracked in three-dimensional space using various modes of detection and types of detectors 34 as well as a processing device 36 which is part of the CAS system 20.

In a first tracking scheme, the configuration of the detectable element 32 is detectable by the CAS system 20 using detectors 34 in order to measure a position and an orientation of the trackable member 26 in time.

Various tracking and communication schemes are possible. For example, communication between the detectable element 32 and the detectors 34 can be performed using various types of signals: optical (active or passive), radio-frequency, microwave, electromagnetic or any other kind of signal allowing for a remote positioning of the trackable member 26 in space.

The detectable element 32 can thus have a set of reflecting devices for reflecting part of the interrogation signal transmitted by a transmitter of the CAS system which is typically part of the detectors 34 themselves. The reflecting devices are typically set up in a geometry that is recognized by the computer-assisted surgery system.

Alternatively, the detectable element 32 can be a transceiver or a simple emitter for emitting a signal which can be detected by the detectors 34 in a way to locate the detectable element 32 in space.

Since the trackable member 26 is affixed to the engaging member 22, which follows movement of the bone element of the body part 24, a position and an orientation of points related to the bone element itself are calculated as a function of the measured position and orientation of the trackable member 26.

The detectable element 32 may have at least three detectable spheres or disks 38 forming a scalene triangle arrangement. Such an arrangement of the spheres or disks permits the precise calculation of a position and orientation of the trackable member 26. According to an embodiment, the detectable element 32 uses the technology described in United States Patent Application Publication No. 20070100325 (Jutras et al.), by the present assignee.

In an embodiment, and as illustrated, the first end of the adjustable support member 28 can be made to substantially extend from the center of the scalene-triangle arrangement. Other arrangements where the adjustable support member 28 extends from any point on the trackable member 26 are also possible.

FIG. 3 illustrates the surgical reference device 10 while in use and engaged on soft tissue of a patient's body part 24, and where the engaging member 22 functions as a foot holder mounted to a support table.

In this case, the engaging member 22 is a support for a heel of a foot 24 of a patient. In other cases, the engaging member 22 can be affixed to another device which engages on the soft tissue of the body part. For example, the engaging member 22 can be affixed to the bed of the patient, to the support table or its rails, to a heavy base or a heavy bag, or to any combination thereof. In such cases, however, the device(s) to which the engaging member 22 is affixed ensures that substantially no relative movement is possible between the engaging member 22 and a bone element defining the body part.

The foot holder 22 can be connected to the support table by a lockable translational joint 42 and a lockable rotational joint 43, whereby the position and orientation of the foot holder 22 is adjustable with respect to the support table. As illustrated in FIG. 3, the translation joint 42 has a track 42, as illustrated. The foot holder 22 is engaged onto soft tissue of the body part 24 such that no relative movement is made possible between the foot holder 22 and any bone elements in the body part 24 which is supported and engaged by the foot holder 22.

The foot holder 22 is locked in a position along the track of the translational joint 42, thereby further blocking the foot and limb 24 in a fixed position.

The embodiment illustrated by FIG. 3 illustrates that the engaging member 22 can be a support device for an articulation, such as a foot, a knee or an elbow holder, for example, which substantially stops any relative movement between the holder and bone elements forming part of the articulation of the patient. The engaging device 22 is also made to block any movement of the articulation to which it is engaged.

Hence, the engaging (or engagement) member 22 can be made to be secured not only on a heel, but also on a knee, a hip, a shoulder, an elbow and the like.

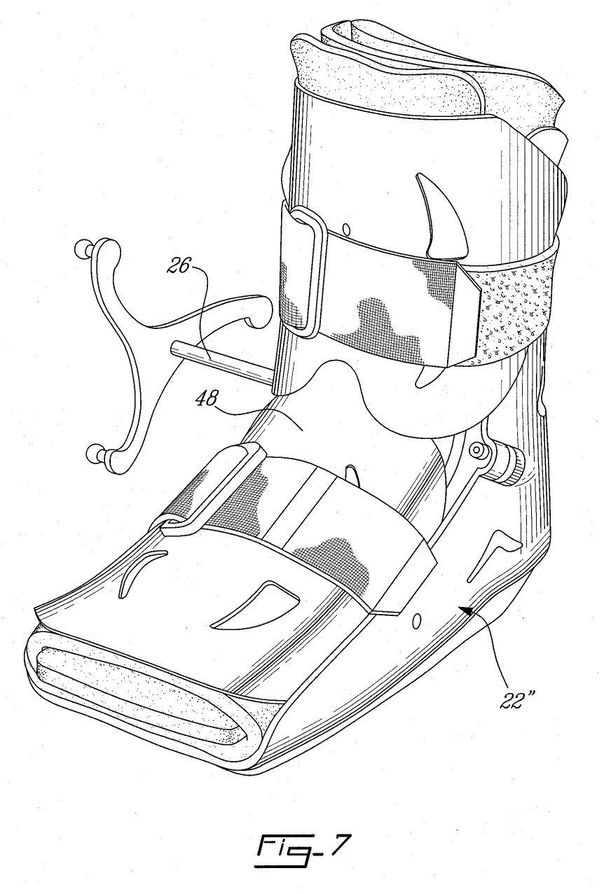

Referring to FIG. 6, an embodiment of the foot holder is illustrated at 22′. The foot holder 22′ has a pair of malleoli contacting members 44 (one of which is visible in FIG. 6) that press against the opposite malleoli of the leg. As is illustrated in FIG. 6, the distance between the malleoli contacting members 44 is adjustable by one or more threaded rods 46 to ensure that the foot holder 22′ is clamped to the leg. As an alternative to a threaded rod, quick-release levers or the like can be used. As is shown in FIG. 6, means are provided to abut against the tibia of the patient. More specifically, a strap 47 is positioned above the malleoli contacting members 44 so as to press the diaphysis of the tibia against a shell of the foot holder 22′. Alternatives to the strap 47 may be used.

Referring to FIG. 7, another embodiment of the foot holder is illustrated at 22″. The foot holder 22″ has one or more inflatable cushions 48 or like inflatable member within a rigid shell of the foot holder 22″ (i.e., an air cast boot). The cushions 48 are inflated so as to immobilize the shell with respect to the tibia. The trackable member 26 is secured to the rigid shell. It is contemplated to provide the foot holder 22″ with a manually actuated pump with a deflating valve, to facilitate the securing of the foot holder 22″ to the tibia, and its subsequent release. For example, the air cast described as SP Walker™ (www.aircast.com) can be used as the foot holder 22″.

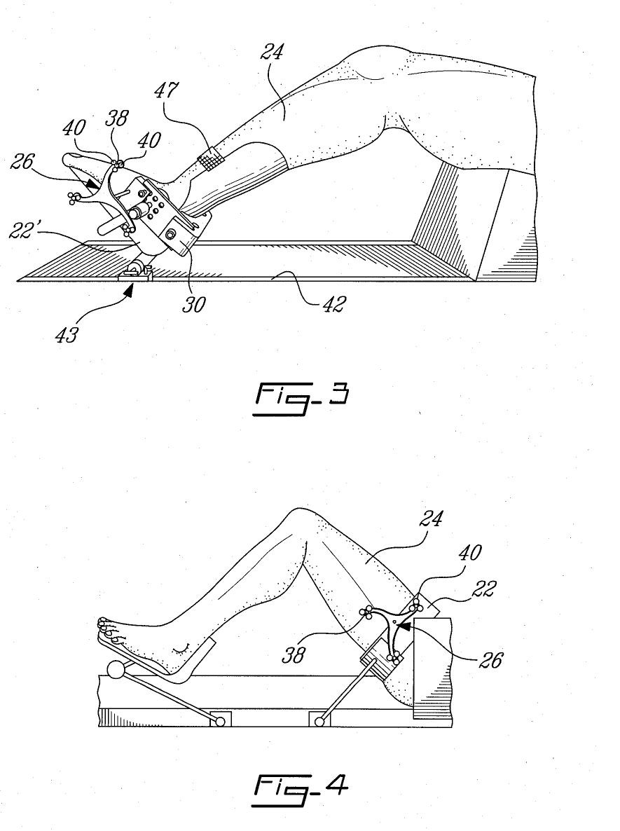

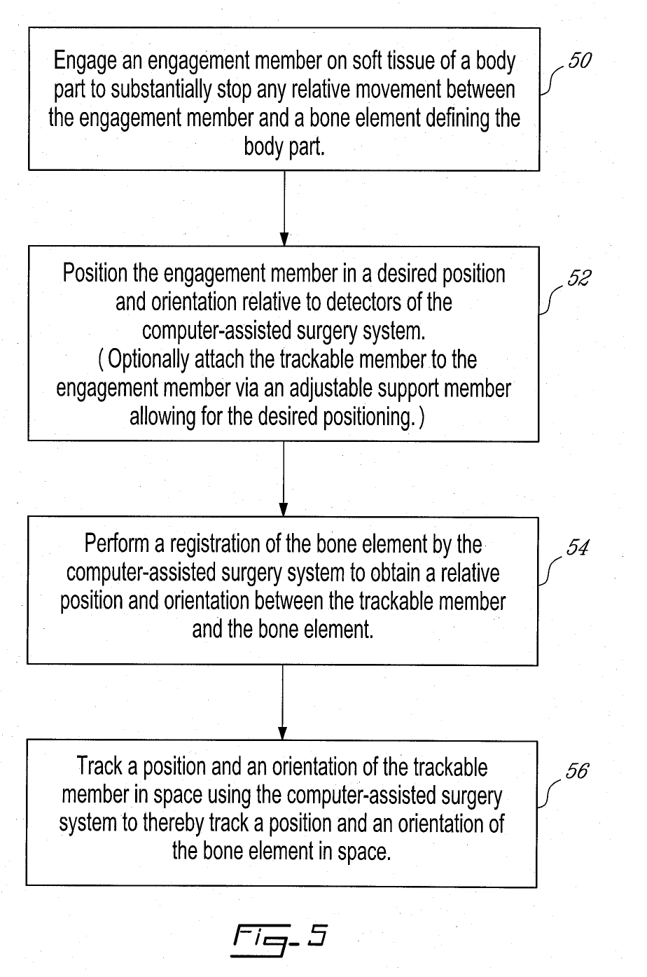

FIG. 4 illustrates the surgical reference device 10 in use on a patient's body part 24, and where the engaging member 22 is a compression bandage.

In this case, the engaging member 22 compresses around the soft tissue of the body part 24 so as to remain in a fixed position with respect to a bone element forming part of the body part 24.

For illustration purposes, the body part in FIG. 4 is a leg of a patient. A body part can however be any other body member whereby the soft tissue of the body member can be compressively bandaged to substantially stop any relative movement between a bone element defining the body member and a trackable member 26 attached to the bandage 22. Such body members can be a foot, a knee, a hip, a shoulder, an arm, forearm, an elbow and the like.

In an embodiment, the engaging member 22 is a strap that is secured to the soft tissue of the patient. One contemplated system for securing the strap 22 is Velcro™ strips. It is considered to secure the trackable member 26 to the strap 22 by a rigid structure, to ensure that there is no movement between the bone element and the trackable member 26. In another embodiment, an adhesive is used to secure the rigid structure (e.g., plate) to the soft tissue to further increase the stability between the trackable member 26 and the bone element through the soft tissue.

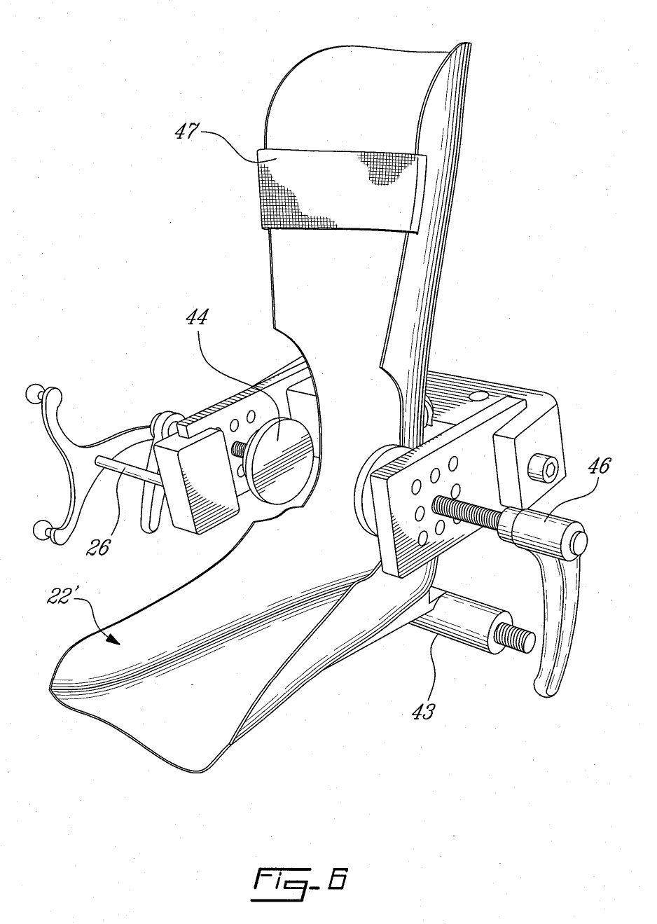

FIG. 5 illustrates a method for monitoring a position and an orientation of a bone element in time using a CAS system.

In step 50, an engaging member is directly secured onto soft tissue of a body part. The engaging member can optionally be secured such as to engage partially around the body part of a patient. In any case, the engaging member is secured such that substantially no relative movement is possible between the engaging member and a bone element defining the body part.

This ensures that the bone element moves with the engaging member 22, whereby a trackable member affixed to the engaging member 22 moves concurrently with the bone element, such that a position and an orientation of the trackable member 26 changes with that of the bone element.

In step 52, the engaging member is positioned on the soft tissue, about the body part and such that the trackable member 26 is in a desired position and orientation relative to detectors of the computer-assisted surgery system. Step 52 is as a function of the type of foot holder used (e.g., any one of the foot holders of FIGS. 2 to 4 and 6-7).

Referring to FIG. 2, the trackable member 26 may be attached to the engagement member 22 via the optional adjustable support member 28. The trackable member 26 can be attached to the optional adjustable support member 28 in a desired position and orientation relative to detectors 34 of the computer assisted surgery system.

Referring to FIGS. 2 and 5, in step 54, a registration of the bone element is performed with respect to the trackable member.

The computer-assisted surgery system 20 is used to track a position and an orientation of the trackable member and relate points of an exposed portion of the bone element thereto in order to obtain a relative measurement between the trackable member 26 and the bone element. This measurement is used by the system to calculate a position and an orientation of the bone element using a measured position and an orientation of the trackable member 26.

Referring to FIGS. 2 and 5, in step 56, the computer-assisted surgery system 20 tracks the position and the orientation of the trackable member 26 in space using its detectors 34. Hence, by tracking the trackable member 26, the position and the orientation of the bone element is also tracked during surgical procedure. The operator therefore obtains position and orientation data pertaining to the bone element, from the tracking of the trackable member 26 and from the registration.

The trackable member 26 can be removed from the engaging member 22 and affixed to another engaging member on another body part, intraoperatively or extra-operatively. The trackable member 26 can also be replaced by another trackable member or removed and be sterilized intraoperatively as needed.

Alternatively, the trackable member 26 can be removed from the body part by removing the engaging member 22 and repositioning the engaging member about the body part or about another body part of the patient, intraoperatively or extraoperatively.

1. A surgical reference device for use with a computer-assisted surgery system, the surgical reference device comprising:
an engaging member adapted to engage directly on soft tissue of a body part such that substantially no relative movement is possible between the engaging member and a bone element of the body part; and
a trackable member attached to the engaging member, the trackable member comprising a detectable element for being located and tracked in three-dimensional space by the computer-assisted surgery system to measure a position and an orientation of the trackable member in time;
whereby the bone element is tracked as a function of the position and orientation of the trackable member.

2. The surgical reference device according to claim 1, wherein the body part is a foot of a patient and the engaging member is a foot holder adapted to receive the foot of the patient, the trackable member being secured to the foot holder.

3. The surgical reference device according to claim 2, wherein the foot holder has a pair of malleoli contacting members adapted to releasably abut against opposite malleoli of the foot to immovably secure the foot holder to the foot of the patient.

4. The surgical reference device according to claim 3, wherein the foot holder has means to abut against a diaphysis of a tibia of the patient to immovable secure the foot holder to the tibia of the patient.

5. The surgical reference device according to claim 3, wherein at least one of the malleoli contacting members is mounted to a shell of the foot holder by a threaded rod allowing translation displacement of the malleoli contacting member.

6. The surgical reference device according to claim 2, wherein the foot holder has at least one inflatable member adapted to immovably secure the foot between an outer shell of the foot holder and the inflatable member.

7. The surgical reference device according to claim 1, wherein the engaging member is a strap strapped to the body part, the strap being secured by Velcro™ strips.

8. The surgical reference device according to claim 7, wherein the body part is a thigh of the patient, and the strap is adapted to be immovably strapped to the thigh of the patient.

9. The surgical reference device according to claim 1, wherein the trackable member is connected to an engaging member by a lockable cylindrical joint.

10. A combination of a surgical reference device and of a support table, comprising:
the surgical reference device in accordance with claim 2;
a support table adapted to support the client; and
at least one lockable joint between the surgical reference device and the support table to adjust at least one of a position and an orientation of the surgical reference device with respect to the table.

11. The combination of claim 10, wherein the at least one joint comprises a lockable rotational joint and a lockable translational joint such that a position and an orientation of the surgical reference device is adjustable with respect to the support table.

12. A method for tracking a position and an orientation of a bone element using a computer-assisted surgery system, the method comprising:
securing an engaging member directly on soft tissue of a body part such that substantially no relative movement is possible between the engaging member and a bone element of the body part, the engaging member having a trackable member thereon;
performing a registration of an exposed portion the bone element with respect to the trackable member; and
obtaining position and orientation data for at least the exposed portion of the bone element as a function of the tracking of a position and an orientation of the trackable member and the registration.

13. The method according to claim 12, wherein securing an engaging member directly on soft tissue of a body part comprises inserting a foot of the user into a foot support and securing the foot to the foot support.

14. The method according to claim 13, wherein securing the foot to the foot support comprises abutting contacting members against the malleoli of the patient.

15. The method according to claim 14, wherein securing the foot to the foot support further comprises securing the foot support against a diaphysis of the tibia.

16. The method according to claim 13, wherein securing the foot to the foot support comprises inflating an inflatable member such that the foot is secured between the inflatable member and the foot support.

17. The method according to claim 12, wherein securing an engaging member directly on soft tissue of a body part comprises strapping the engaging member against a thigh of the patient.

18. The method according to claim 13, further comprising adjusting at least one of a position and an orientation of the foot holder with respect to a support table by adjusting at least one joint between the foot holder and the support table, prior to performing the registration, for the trackable member to be detectable by the computer-assisted surgery system.

19. The method according to claim 12, further comprising adjusting at least one of a position and an orientation of the trackable member with respect to the engaging member prior to performing the registration, for the trackable member to be detectable by the computer-assisted surgery system.
